(19) United States
(12) Patent Application Publication
(10) Pub. No.: US 2016/0029605 A1
(43) Pub. Date: Feb. 4, 2016

(54) METHOD FOR CONTROLLING LASIODERMA SERRICORNE USING ANISOPTEROMALUS APIOVORUS

(71) Applicant: CHANGWON NATIONAL UNIVERSITY Industry Academy Corporation Corps, Gyeongsangnam-do (KR)

(72) Inventors: Seon Woo CHEONG, Gyeongsangnam-do (KR); Chun Sik YOON, Gyeongsangnam-do (KR)

(21) Appl. No.: 14/491,975

(22) Filed: Sep. 19, 2014

(30) Foreign Application Priority Data
Jul. 29, 2014 (KR) 10-2014-0096180

(51) Int. Cl. A01K 67/033; A01M 99/00

(52) U.S. Cl. CPC A01K 67/033; A01M 99/00

(57) ABSTRACT

A method for the biological control of Lasioderma serricorne and a method for the mass rearing of Anisopteromalus apiovorus are disclosed. The method for the biological control of Lasioderma serricorne includes introducing Anisopteromalus apiovorus, which is a natural enemy of Lasioderma serricorne, to a source of Lasioderma serricorne. The method for the mass rearing of Anisopteromalus apiovorus includes inducing Anisopteromalus apiovorus to lay eggs on larvae of Lasioderma serricorne, and allowing the eggs laid by the Anisopteromalus apiovorus to emerge into adult insects.

### CROSS-REFERENCE TO RELATED APPLICATIONS AND CLAIM OF PRIORITY

The present application claims the benefit of Korean Patent Application No. 10-2014-0096180, filed Jul. 29, 2014 in the Korean Intellectual Property Office, the entire contents of which are incorporated herein by reference.

### BACKGROUND

1. Technical Field

The present disclosure relates to a method for controlling Lasioderma serricorne using Anisopteromalus apiovorus and a method for the mass rearing of Anisopteromalus apiovorus.

2. Description of the Related Art

Lasioderma serricorne and Ephestia elutella are known as major insect pests that cause damage to tobacco, and about 0.7% of all tobacco products that are distributed in the USA are lost due to damage caused by the two insect pests (USDA, 1972; Stored tobacco insect-Biology and control-Agriculture handbook No. 233). Of them, Lasioderma serricorne causes damage to tobacco products and tobacco raw materials in all developmental stages thereof, and major damage occurs in the larval stages. Particularly, this insect pest forms small cylindrical tunnels into tobacco products and enters the cylindrical tunnels, and then the cylindrical tunnels are filled with a mass of dust and excrement, thus causing consumer complaints. In addition to tobacco, Lasioderma serricorne also causes damage to grains such as rice, wheat bran and starch, paper, and dried plants such as dried figs, powdered red pepper, ginger, curry powder, raisins, saffron and licorice, as well as dried fishes, clothes and wood (Runner, 1919; The tobacco beetle: An important pest in tobacco product. U.S. Dep. Agric. Cir. 737).

Furthermore, adult Lasioderma serricorne is known to cause damage to food packaging materials by perforating them (Mahroof, 2008; Life history parameters of Lasioderma (F.) as influenced by food sources. Journal of Stored products Research 44(3) 219-226). Moreover, since adult Lasioderma serricorne perforates and invades food packaging materials and propagates therein, the dead bodies and shells of Lasioderma serricorne remain in the food (Cabrera, 2007, Featured Creatures; Entomology & Nematology. Florida Department of Agriculture and Consumer Services. EENY-227. USA). As described above, Lasioderma serricorne lives on a wide range of materials and has an excellent ability to survive, and thus is known as an insect pest that causes serious economic damage.

Since Lasioderma serricorne is closely related to foods or favorite items that are consumed by humans, chemical control methods cannot be used to control Lasioderma serricorne. Methods that use radiation or low-temperature storage can be used to control Lasioderma serricorne, but are difficult to put into practical use because these methods incur high costs. In addition, biological control methods that use natural enemies can be effectively used to control insect pests because they pose no risk of residual pesticides, and thus are harmless to the human body and are environmentally friendly. However, the use of natural enemies in biological control methods is significantly limited because there are currently few insect species having clearly established relationships between insect pests and their natural enemies, and studies on these relationships have been insufficient.

Accordingly, the present inventors have conducted extensive studies to control Lasioderma serricorne, and, as a result, have found that, when Anisopteromalus apiovorus lays eggs on larvae of Lasioderma serricorne and the laid eggs hatch and parasitize the larvae of Lasioderma serricorne, Lasioderma serricorne will be very effectively controlled, and also have elucidated the mechanism of biological control of the insect pest Lasioderma serricorne by its natural enemy Anisopteromalus apiovorus, resulting in the completion of the present invention.

### SUMMARY

At least one embodiment of the present disclosure is intended to provide a method for controlling Lasioderma serricorne using Anisopteromalus apiovorus, which is harmless to the human body and can control Lasioderma serricorne in an environmentally friendly and effective way.

At least one embodiment of the present disclosure is intended to provide a method for mass rearing of Anisopteromalus apiovorus using Lasioderma serricorne larvae, which is used in the method for controlling Lasioderma serricorne.

In accordance with an aspect of the present disclosure, there is provided a method for biological control of Lasioderma serricorne, the method including introducing Anisopteromalus apiovorus, which is a natural enemy of Lasioderma serricorne, to the source of Lasioderma serricorne.

Anisopteromalus apiovorus that is used in the present disclosure refers to parasitic wasps whose females lay eggs on larvae of other insects to propagate offspring. It was a new species native to the African continent, and was reported in 1988. The distribution of this insect in areas other than the African continent has not yet been reported. Although Anisopteromalus apiovorus has not yet been reported in Korea, the present inventors have found that Anisopteromalus apiovorus is different in the antenna funicle length from the allied species Anisopteromalus calandrae, and has a body length of about 2-2.8 mm, a golden black color throughout the body, and yellowish-white hair that grown thick, and thus it is the same as described in the original description (Rasplus, 1988 Bullutin de la Societe Entomologique de France, 93, 119-127). Also, based on the results of analysis of the mitochondrial CO1 gene, the present inventors have demonstrated that Anisopteromalus apiovorus is a species different from the allied species Anisopteromalus calandrae. In addition, it was found that Anisopteromalus apiovorus was parasitic on Lasioderma serricorne as its host and thus the present inventors named it in Korean “Kwon-yeon-beol-le-sa-ri-geum-zom-beol.”

In the present disclosure, the source of Lasioderma serricorne may be any habitat, such as processed food, dried agricultural products, tobacco or wood, in which Lasioderma serricorne can live. Thus, the source of Lasioderma serricorne is not specifically limited. The source of Lasioderma serricorne may be selected from the group consisting of grains, processed grain products, stored tobacco, processed tobacco products, zoological/botanical specimens, herbal medicines, herbs, spice, dried fishes, wood, paper, a facility for storing these items, and a facility for processing these items.

In the present disclosure, Anisopteromalus apiovorus exhibits characteristics in that it lays eggs on larvae of Lasioderma serricorne and the laid eggs hatch and is parasitic on Lasioderma serricorne.

In view of the efficiency of control, the number of Anisopteromalus apiovorus introduced per individual of Lasioderma serricorne is preferably 0.01 to 0.1, and more preferably 0.038 to 0.043.

In an embodiment of the present disclosure, it was found that, when 5 individuals of Anisopteromalus apiovorus were introduced to an experimental group having a host density of 150 individuals (Lasioderma serricorne), the control rate of the hosts was 98.4%, and when 3 individuals of Anisopteromalus apiovorus were introduced to an experimental group having a host density of 75 individuals (Lasioderma serricorne), the control rate of the hosts was 98.2%. From these results, it can be seen that, in the experimental group having a host density of 150 individuals (Lasioderma serricorne), about 26 host individuals per individual of Anisopteromalus apiovorus can be controlled, and thus 0.038 individuals of Anisopteromalus apiovorus are required to control one individual of Lasioderma serricorne. In addition, in the experimental group having a host density of 75 individuals (Lasioderma serricorne), about 23 host individuals per individual of Anisopteromalus apiovorus can be controlled, and thus 0.043 individuals of Anisopteromalus apiovorus are required to control one individual of Lasioderma serricorne.

In another aspect of the present disclosure, there is provided a method for mass rearing of Anisopteromalus apiovorus, the method including inducing Anisopteromalus apiovorus to lay eggs on larvae of Lasioderma serricorne; and allowing the eggs laid by the Anisopteromalus apiovorus to emerge into adult insects.

In the present disclosure, inducing the Anisopteromalus apiovorus to lay eggs and allowing the laid eggs to emerge may be both performed at a temperature of 28 to 38° C., preferably 30 to 36° C., and more preferably 34±0.5° C.

In an embodiment of the present disclosure, in order to measure the development rate of Anisopteromalus apiovorus in each developmental stage at varying temperatures, the developmental stage of Anisopteromalus apiovorus was observed at 24-hour intervals while Anisopteromalus apiovorus was stored at temperatures of 18, 20, 22, 24, 26, 28, 30, 32, 34, 36 and 38±0.5° C. and a relative humidity of 70 to 75% under a 12-hr light/12-hr dark cycle, and, as a result, it was found that the optimum temperature that exhibited the shortest life cycle is 34±0.5° C.

### BRIEF DESCRIPTION OF THE FIGURES

The above and other objects, features and advantages of the present disclosure will be more clearly understood from the following detailed description taken in conjunction with the accompanying figures, in which:

FIG. 1 is a view illustrating a difference in antenna between Anisopteromalus apiovorus and Anisopteromalus calandrae;

FIG. 2 is a view showing the comparison of the nucleotide sequence of mitochondrial CO1 gene between Anisopteromalus apiovorus and Anisopteromalus calandrae;

FIG. 3 is a view illustrating that Anisopteromalus apiovorus lays eggs on larvae of Lasioderma serricorne;

FIG. 4 is a view illustrating that eggs laid on larvae of Lasioderma serricorne are parasitic on the larvae after hatching;

FIG. 5 is a graph showing the number of eggs laid by Anisopteromalus apiovorus as a function of the age of adult insects of Anisopteromalus apiovorus; and

FIG. 6 is a graph showing the effects of Anisopteromalus apiovorus on the biological control of Lasioderma serricorne.

### DETAILED DESCRIPTION

The present disclosure will be described in detail below with reference to examples. It is to be appreciated, however, that these examples are for illustrative purposes only and are not intended to limit the scope of the present disclosure.

### Example 1

### Taxonomic Examination of Anisopteromalus apiovorus

In order to distinguish Anisopteromalus apiovorus from other allied species, the following experiment was performed.

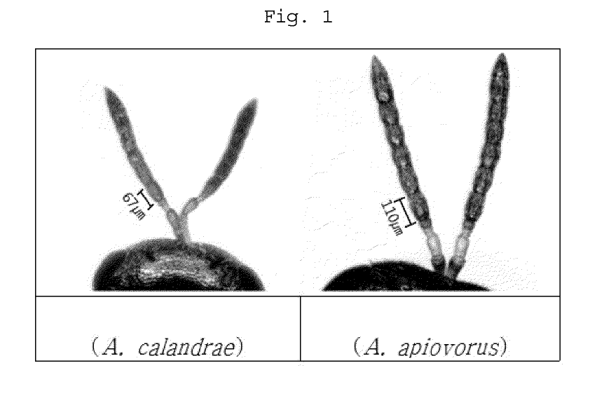

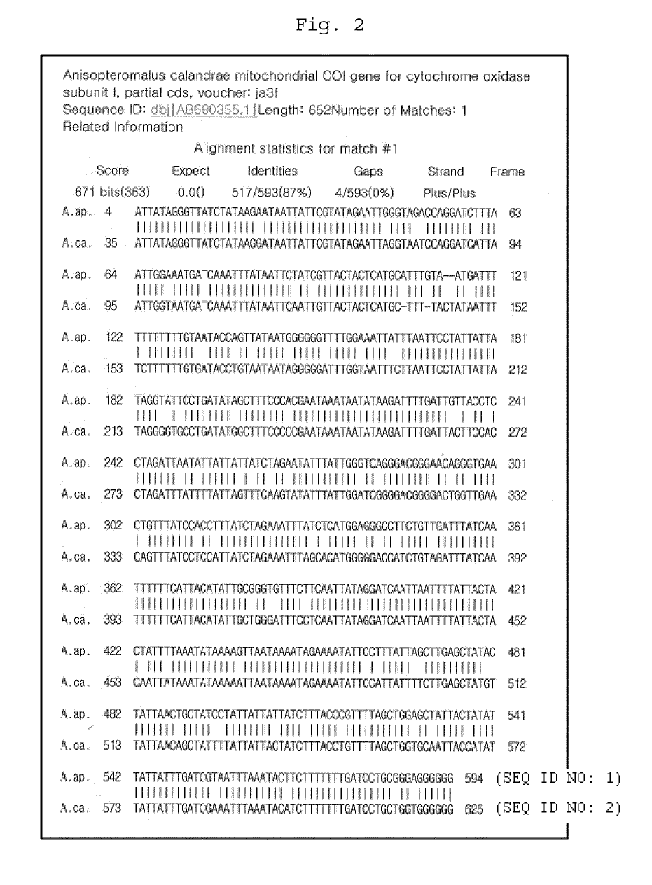

In November 2012, a wasp parasitic on Lasioderma serricorne as its host was found in Gyeongsangnam-do, Korea, and collected using an insect net and a suction tube aspirator. The collected parasitic wasp was compared with allied species and the original description (Rasplus, 1988 Bullutin de la Societe Entomologique de France, 93, 119-127) to examine whether it was reported in Korea. In addition, the morphological characteristics of the collected parasitic wasp were observed using a stereoscopic microscope (Zeiss Stemi SV 11 Apo). Also, the mitochondrial CO1 gene was analyzed by a molecular biological method, and, as a result, it was demonstrated that Anisopteromalus apiovorus is a specific different from the allied species Anisopteromalus calandrae distributed in Korea. The collected parasitic wasp had a body length of about 2 to 2.8 mm, a golden black color throughout the body, and yellowish white hair that grown thick. As shown in FIG. 1, Anisopteromalus apiovorus had a difference in antenna funicle length from the allied species Anisopteromalus calandrae distributed in Korea. In addition, as shown in FIG. 2, the nucleotide sequence of the mitochondrial CO1 gene of Anisopteromalus apiovorus had an identity of 87% with that of Anisopteromalus calandrae, suggesting that it was a species different from Anisopteromalus calandrae. Because the collected parasitic wasp was a species of the genus Anisopteromalus of the family Pteromalidae, had not yet reported in Korea and was parasitic on Lasioderma serricorne as its host, it was named in Korean “Kwon-yeon-beol-le-sa-ri-geum-zom-beol.”

### Example 2

### Conditions for Rearing of Anisopteromalus apiovorus

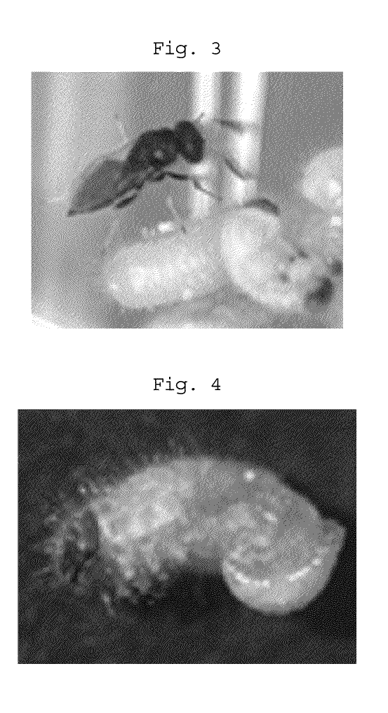

In a plastic cage for insects (100 mm diameter×40 mm height), last-stage larvae of Lasioderma serricorne having a head capsule width of 0.65±0.05 mm were used as hosts. More specifically, 50 individuals of Lasioderma serricorne and 5 g of artificial feed were placed in a plastic cage, and then 4 pairs of female and male Anisopteromalus apiovorus within 24 hours after emergence were placed in the cage and induced to lay eggs on larvae of the Lasioderma serricorne for 120 hours at a temperature of 30±0.5° C. and a relative humidity of 70 to 75% under a 12-hr light/12-hr dark cycle. The Anisopteromalus apiovorus that laid eggs was removed from the cage, and the cage was maintained under the same environmental conditions as above, after which the emerged parasitic Anisopteromalus apiovorus individuals were separated and collected at 24-hr intervals. FIG. 3 illustrates that Anisopteromalus apiovorus lays eggs on Lasioderma serricorne larvae, and FIG. 4 illustrates that the laid eggs are parasitic on Lasioderma serricorne after hatching.

### Example 3

### Measurement of Developmental Rate in Each Developmental Stage at Varying Temperatures

100 last-stage larvae of Lasioderma serricorne were placed in an insect breeding dish (120 mm diameter×80 mm height) which was then placed in an acrylic cage (300 mm width×300 mm length×300 mm height). Next, 10 individuals of female Anisopteromalus apiovorus were placed in the cage and induced to lay eggs on the Lasioderma serricorne larvae. After 24 hours, the insect breeding dish was taken out of the cage, and Lasioderma serricorne larvae having Anisopteromalus apiovorus eggs laid thereon were separated from the insect breeding dish under a stereoscopic microscope. Each of 20 Lasioderma serricorne larvae having Anisopteromalus apiovorus eggs laid thereon was placed in each well of a 24-well plate which was then covered. Next, the Lasioderma serricorne larvae were stored at temperatures of 18, 20, 22, 24, 26, 28, 30, 32, 34, 36 and 38±0.5° C. and a relative humidity of 70-75% under a 12-hr light/12-hr dark cycle while the time (days) taken for the Anisopteromalus apiovorus to develop into eggs, larvae, pupae and adult insects was observed at 24-hr intervals. After emergence into adult insects, the individuals were classified into male and female. In addition, the experiment for the measurement of developmental rate at each temperature was repeated four times. The results of the measurement are shown in Table 1 below.

TABLE 1

| Temperature | | | | | | |
|---|---|---|---|---|---|---|
| (° C.) | N | Sex | Egg | Larva | Pupa | Total |
| 18 | 31 | F | 5.323 ± 0.653 | 15.741 ± 2.556 | 20.419 ± 1.098 | 41.484 ± 3.140 |
| | 7 | M | 5.571 ± 0.535 | 17.286 ± 1.604 | 19.714 ± 2.059 | 42.571 ± 3.867 |
| 20 | 36 | F | 3.944 ± 1.827 | 14.889 ± 1.897 | 17.861 ± 0.672 | 36.694 ± 1.827 |
| | 8 | M | 4.125 ± 0.354 | 14.625 ± 1.302 | 17.625 ± 0.744 | 36.638 ± 1.733 |
| 22 | 37 | F | 3.316 ± 0.435 | 11.895 ± 1.381 | 14.263 ± 0.684 | 29.474 ± 1.827 |
| | 9 | M | 3.400 ± 0.516 | 11.600 ± 1.430 | 13.400 ± 1.075 | 28.400 ± 1.955 |
| 24 | 35 | F | 2.486 ± 0.507 | 9.829 ± 1.723 | 13.029 ± 0.664 | 25.343 ± 2.195 |
| | 18 | M | 2.389 ± 0.507 | 9.278 ± 1.121 | 12.611 ± 0.870 | 24.278 ± 1.770 |
| 26 | 30 | F | 1.655 ± 0.485 | 8.655 ± 0.836 | 11.621 ± 1.107 | 21.931 ± 1.297 |
| | 18 | M | l.833 ± 0.383 | 6.889 ± 1.023 | 10.889 ± 1.132 | 21.611 ± 1.290 |
| 28 | 43 | F | 1.442 ± 0.054 | 6.628 ± 0.914 | 9.907 ± 0.842 | 17.977 ± 1.070 |
| | 7 | M | 1.286 ± 0.488 | 6.714 ± 0.951 | 9.714 ± 0.488 | 17.714 ± 1.254 |
| 30 | 42 | F | 1.268 ± 0.435 | 6.038 ± 1.221 | 8.707 ± 0.618 | 16.073 ± 1.321 |
| | 14 | M | 1.286 ± 0.469 | 6.143 ± 1.292 | 8.714 ± 0.611 | 16.143 ± 1.460 |
| 32 | 38 | F | 1.079 ± 0.232 | 5.316 ± 0.577 | 8.263 ± 0.604 | 14.658 ± 0.871 |
| | 18 | M | 1.278 ± 0.575 | 6.111 ± 2.026 | 7.833 ± 0.924 | 15.222 ± 2.211 |
| 34 | 24 | F | 1.083 ± 0.282 | 5.125 ± 0.797 | 8.375 ± 0.576 | 14.583 ± 0.766 |
| | 20 | M | 1.150 ± 0.351 | 5.650 ± 0.854 | 7.900 ± 0.617 | 14.700 ± 0.767 |
| 36 | 23 | F | 1.174 ± 0.388 | 5.826 ± 0.650 | 8.130 ± 0.626 | 15.130 ± 0.968 |
| | 11 | M | 1.182 ± 0.405 | 6.182 ± 0.405 | 8.182 ± 0.603 | 15.545 ± 0.934 |
| 38 | 25 | F | 1.360 ± 0.490 | 6.840 ± 1.375 | 8.560 ± 0.768 | 16.760 ± 1.615 |
| | 6 | M | 1.657 ± 0.516 | 6.833 ± 0.753 | 8.333 ± 0.816 | 16.833 ± 1.329 |

As can be seen in Table 1 above, at a temperature ranging from 18° C. to 26±0.5° C., the time it takes for eggs to emerge into adult insects was about 20 to 40 days, and at a temperature higher than 28° C., the emergence time was shorter. Also, it was found that the optimum temperature that exhibited the shortest life cycle was 34±0.5° C.

### Example 4

### Measurement of Average Lifespan of Anisopteromalus apiovorus and Number of Eggs Laid by Anisopteromalus apiovorus

Anisopteromalus apiovorus was reared at a temperature of 30±0.5° C. and a relative humidity of 70 to 75% under a 12-hr light/12-hr dark cycle as described in Examples 2 and 3, and the average lifespan of the reared females and males was measured. Also, the number of eggs laid by female Anisopteromalus apiovorus during 1-18 days after mating with male Anisopteromalus apiovorus was measured. As a result, it was found that the average lifespan was 14.4±3.6 days for females and 9.1±1.2 days for males.

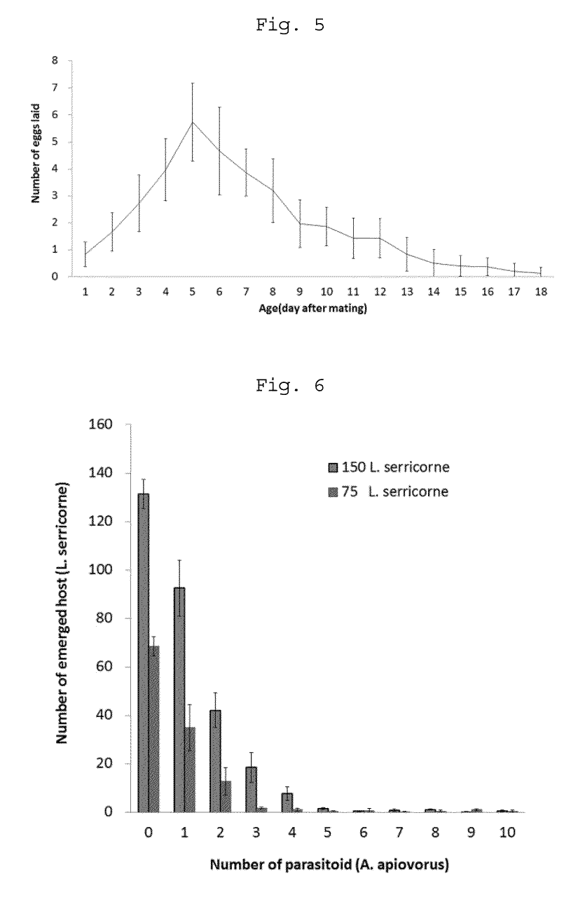

In addition, as shown in FIG. 5, the total number of eggs laid by female Anisopteromalus apiovorus was 35.1±13.5, and the number of eggs laid increased gradually from 0.8±0.9 on day 1 to 1.7±1.4 on day 2, 2.7±2.1 on day 3 and 4.0±2.32 on day 4 and reached the peak (5.7±2.9) on day 5. Also, it was found that the number of eggs laid decreased gradually from 4.7±3.3 on day 6 to 3.9±1.7 on day 7, to 3.2±2.4 on day 8, to 2.0±1.8 on day 9, and to 1.3±1.2 on day 10.

### Example 5

### Effects on Control of Lasioderma serricorne

Last-stage larvae of Lasioderma serricorne were divided into two groups: one group consisting of 150 individuals, and another group consisting of 75 individuals. The Lasioderma serricorne larvae of each group were placed in an insect breeding dish (120 mm diameter×80 mm height) together with 10 g or 7.5 g of artificial feed, and the uncovered dish of each group was placed in an acrylic cage for insects (300 mm width×300 mm length×300 mm height), after which males and females of Anisopteromalus apiovorus were mated with one another. Within 24 hours after mating, 0-10 individuals of the female Anisopteromalus apiovorus were introduced to the Lasioderma serricorne of the two groups at 10 different introduction densities, and were then stored at a temperature of 30±0.5° C. and a relative humidity of 70 to 75% under a 12-hr light/12-hr dark cycle. The emerged hosts were separated and counted at 24-hour intervals until the hosts no longer emerged, and the experiment was repeated five times. The results of the experiment are shown in Table 2 below and FIG. 6.

TABLE 2

| N. | N. of | | | | | | | | | | | |
|---|---|---|---|---|---|---|---|---|---|---|---|---|
| of | L. serri- | Number of Female A. apiovorus | | | | | | | | | | |
| Exp. | corne | 0 | 1 | 2 | 3 | 4 | 5 | 6 | 7 | 8 | 9 | 10 |
| 1 | 150 | 112 | 62 | 23 | 14 | 5 | 2 | 1 | 2 | 1 | 1 | 0 |
| | 75 | 58 | 22 | 6 | 2 | 0 | 1 | 4 | 0 | 0 | 2 | 0 |
| 2 | 150 | 138 | 124 | 38 | 22 | 6 | 1 | 1 | 0 | 2 | 0 | 2 |
| | 75 | 65 | 24 | 6 | 3 | 2 | 0 | 0 | 1 | 1 | 0 | 0 |
| 3 | 150 | 133 | 82 | 62 | 17 | 3 | 3 | 0 | 2 | 1 | 0 | 0 |
| | 75 | 71 | 58 | 17 | 2 | 1 | 0 | 0 | 0 | 2 | 2 | 1 |
| 4 | 150 | 130 | 104 | 46 | 3 | 18 | 1 | 1 | 1 | 1 | 0 | 1 |
| | 75 | 69 | 53 | 31 | 1 | 0 | 0 | 0 | 0 | 0 | 1 | 0 |
| 5 | 150 | 144 | 91 | 42 | 37 | 7 | 1 | 0 | 0 | 1 | 0 | 0 |
| | 75 | 80 | 18 | 4 | 1 | 3 | 1 | 0 | 0 | 0 | 1 | 2 |

As can be seen in Table 2 above and FIG. 6, when 5 individuals of Anisopteromalus apiovorus were introduced to the experimental group having a host density of 150 individuals, the control rate of the hosts (Lasioderma serricorne) was 98.4%; when 3 individuals of Anisopteromalus apiovorus were introduced to the experimental group having a host density of 75 individuals, the control rate of the hosts (Lasioderma serricorne) was 98.2%. From these results, it can be seen that, in the experimental group having a host density of 150 individuals (Lasioderma serricorne), About 26 host individuals per individual of Anisopteromalus apiovorus can be controlled, and thus 0.038 individuals of Anisopteromalus apiovorus are required to control one individual of Lasioderma serricorne; and in the experimental group having a host density of 75 individuals (Lasioderma serricorne), about 23 host individuals per individual of Anisopteromalus apiovorus can be controlled, and thus 0.043 individuals of Anisopteromalus apiovorus are required to control one individual of Lasioderma serricorne.

Taken together, it was found that the suitable ratio of the number of individuals of Anisopteromalus apiovorus, which were used to control Lasioderma serricorne, to the number of the host Lasioderma serricorne was in the range from 1:0.038 to 1:0.043, and the number of individuals of Lasioderma serricorne that could be controlled by one individual of Anisopteromalus apiovorus was 23-26, proving that Anisopteromalus apiovorus was a natural enemy insect that was very effective in controlling Lasioderma serricorne.

As described above, in accordance with to the present disclosure, Lasioderma serricorne that is native to Egypt is an economically harmful insect pest that easily propagates under a high-temperature environment, and Anisopteromalus apiovorus that is a thermophilic insect native to Africa is a very effective natural enemy of Lasioderma serricorne. Particularly, Anisopteromalus apiovorus has excellent effects on the control of Lasioderma serricorne that causes serious damage to economically highly valuable materials, such as grains or tobacco, when these valuable materials are stored at room temperature or high temperatures. Anisopteromalus apiovorus is parasitic on Lasioderma serricorne as its host and cannot proliferate in the absence of Lasioderma serricorne, and thus it is a good natural enemy that does not cause secondary environmental problems resulting from the prevalence of a natural enemy of Lasioderma serricorne after the control of Lasioderma serricorne. In addition, it was found that the number of individuals of Lasioderma serricorne, which could be controlled by one individual of Anisopteromalus apiovorus after introduction, was about 23-26, proving that Anisopteromalus apiovorus had distinct effects on the control of Lasioderma serricorne. Accordingly, the control method of the present disclosure can be used in various fields to effectively control Lasioderma serricorne.

Although the specific embodiments of the present disclosure have been disclosed for illustrative purposes, those skilled in the art will appreciate that various modifications, additions and substitutions are possible without departing from the scope and spirit of the disclosure as disclosed in the accompanying claims.

What is claimed is:

1. A method for biological control of Lasioderma serricorne, the method comprising introducing Anisopteromalus apiovorus, which is a natural enemy of Lasioderma serricorne, to a source of Lasioderma serricorne.

2. The method of claim 1, wherein the source of Lasioderma serricorne is selected from the group consisting of grains, processed grain products, stored tobacco, processed tobacco products, zoological/botanical specimens, herbal medicines, herbs, spice, dried fishes, wood, paper, a facility for storing the items, and a facility for processing the items.

3. The method of claim 1, wherein the Anisopteromalus apiovorus lays eggs on larvae of the Lasioderma serricorne, and the laid eggs hatch and are parasitic on the Lasioderma serricorne.

4. The method of claim 1, wherein a number of Anisopteromalus apiovorus introduced per individual of the Lasioderma serricorne is 0.03 to 0.05.

5. A method for mass rearing of Anisopteromalus apiovorus, the method comprising:
inducing Anisopteromalus apiovorus to lay eggs on larvae of Lasioderma serricorne; and
allowing the eggs laid by the Anisopteromalus apiovorus to emerge into adult insects.

6. The method of claim 5, wherein inducing the Anisopteromalus apiovorus to lay the eggs and allowing the eggs to emerge into the adult insects are both performed at a temperature ranging from 28° C. to 38° C.


SEQUENCE LISTING (2 sequences)

SEQ ID NO: 1 - DNA, 591 bp, Anisopteromalus apiovorus
attatagggttatctataagaataattattcgtatagaattgggtagaccaggatctttaattggaaatgatcaaatttataattctatcgttactactcatgcatttgtaatgatttttttttttgtaataccagttataatggggggttttggaaattatttaattcctattattataggtattcctgatatagctttcccacgaataaataatataagattttgattgttacctcctagattaatattattattatctagaatatttattgggtcagggacgggaacagggtgaactgtttatccacctttatctagaaatttatctcatggagggccttctgttgatttatcaattttttcattacatattgcgggtgtttcttcaattataggatcaattaattttattactactattttaaatataaaagttaataaaatagaaaatattcctttattagcttgagctatactattaactgctatcctattattattatctttacccgttttagctggagctattactatattattatttgatcgtaatttaaatacttctttttttgatcctgcgggagggggg

SEQ ID NO: 2 - DNA, 591 bp, Anisopteromalus calandrae
attatagggttatctataaggataattattcgtatagaattaggtaatccaggatcattaattggtaatgatcaaatttataattcaattgttactactcatgcttttactataattttcttttttgtgatacctgtaataatagggggatttggtaatttcttaattcctattattataggggtgcctgatatgggtttcccccgaataaataatataagattttgattacttccacctagatttattttattagtttcaagtatatttattggatcggggacggggactggttgaacagtttatcctccattatctagaaatttagcacatgggggaccatctgtagatttatcaattttttcattacatattgctgggatttcctcaattataggatcaattaattttattactacaattataaatataaaaattaataaaatagaaaatattccattattttcttgagctatgttattaacagctattttattattactatctttacctgttttagctggtgcaattaccatattattatttgatcgaaatttaaatacatctttttttgatcctgctggtgggggg